(19) United States
(12) Patent Application Publication
(10) Pub. No.: US 2009/0088628 A1
(43) Pub. Date: Apr. 2, 2009

(54) EFFICIENT WORKFLOW FOR AFIB TREATMENT IN THE EP LAB

(76) Inventor: Klaus Klingenbeck-Regn, Nurnberg (DE)

Correspondence Address: BRINKS HOFER GILSON & LIONE, P.O. BOX 10395, CHICAGO, IL, 60610, US

(21) Appl. No.: 11/862,755

(22) Filed: Sep. 27, 2007

(51) Int. Cl. A61B 6/02; A61B 8/12; G06Q 50/00

(52) U.S. Cl. 600/424; 600/407; 600/427; 600/439; 705/2

(57) ABSTRACT

A system and method relate to enhanced medical workflows. Current anatomical data of a patient may be acquired, such as via a computed tomography or magnetic resonance procedure, just before and/or during an intervention. During the intervention, an electroanatomical map of the patient may be generated. The electroanatomical map may be generated from three-dimensional ultrasound data acquired via a medical device. The current anatomical data and electroanatomical map may be dynamically fused during the intervention. The fused data may be displayed and/or used to localize a current position of the medical device in real-time. The medical device may be a catheter employing a multi-dimensional forward-looking array of sensors. In one aspect, the enhanced workflow may be associated with an ablation procedure or other intervention performed in an electrophysiology lab. The use of current anatomical data and more accurate multi-dimensional ultrasound data may alleviate inefficiencies and inaccuracies associated with conventional interventions.

### BACKGROUND

The present embodiments relate generally to the medical treatment of patients. More particularly, the present embodiments relate to enhanced medical workflows and devices used during interventional medical procedures.

Conventional medical workflows may be cumbersome, inefficient, and/or costly. Some workflows may take up to six hours or more. As an example, medical workflows related to the field of electrophysiology (EP) may involve electroanatomical (EA) mapping and the registration of the EA map with anatomical data. The anatomical data may be acquired from pre-interventional computed tomography (CT) or magnetic resonance (MR) individual procedures, which may be time consuming. The electroanatomical mapping itself may require additional time, as well as a dedicated mapping catheter having position sensors integrated into the catheter, such as commercially available carto systems from Biosense Webster.™

Yet, the registration, or integration, of the anatomical map (acquired via CT or MR procedures) with the EA map may be inaccurate. Inaccuracies may arise due to the conventional anatomical data not being up-to-date. The pre-interventional CT/MR scan may have been taken days or weeks before. As a result, conventional anatomical data used during an intervention may not represent the actual, current status of the patient.

Additionally, conventional mapping catheters may not provide for adequate localization of the catheter tip. Therefore, another source of errors associated with the registration of EA maps with anatomical data may be an intrinsic error associated with catheter localization.

### BRIEF SUMMARY

A system and method relate to enhanced medical workflows and devices. The workflow may involve, as part of an interventional medical procedure, (1) acquiring current anatomical data of a patient, and (2) generating an electroanatomical (EA) map of the patient. The anatomical data may be acquired via a computed tomography, magnetic resonance, or other medical imaging procedure. The EA map may be generated from data acquired using a medical device, such as three-dimensional ultrasound data. The anatomical data may be dynamically fused or integrated with the EA map during the intervention. As a result, the fused image data may be displayed and/or used to automatically or visually accurately localize the medical device in real-time during the intervention. In one aspect, the medical device may be an enhanced catheter employing a multi-dimensional looking array of ultrasound sensors. In another aspect, the workflow may be associated with an ablation procedure performed within an electrophysiology (EP) lab. Using current anatomical data in conjunction with more accurate multi-dimensional ultrasound data during an intervention may alleviate inefficiencies and inaccuracies associated with conventional workflows.

In one embodiment, a medical method for assisting, with a system, an interventional procedure is provided. The method includes, as part of the interventional medical procedure, (1) acquiring current anatomical data of a patient via a medical imaging device; (2) generating a current electroanatomical map of the patient; (3) dynamically fusing the current anatomical data with the current electroanatomical map; and (4) displaying fused images associated with the fusion of the current anatomical data with the current electroanatomical map in real-time such that the fused images displayed facilitate completion of the interventional medical procedure.

In another embodiment, a medical method for assisting, with a system, an interventional procedure is provided. The method includes acquiring current anatomical data of a patient, as part of an interventional procedure, when the patient is on a medical table; generating an electroanatomical map of the patient using data acquired from a medical device inserted into the patient during the interventional procedure; dynamically fusing the current anatomical data and the electroanatomical map to create composite images; localizing a position of the medical device within the patient in relation to the composite images; and displaying the composite images associated with the fused current anatomical data and the electroanatomical map such that the localized position of the medical device within the patient during the interventional procedure may be ascertained.

In another embodiment, a data processing system facilitates a interventional workflow. The system includes a processing unit that (1) receives current anatomical data of a patient; (2) receives multi-dimensional ultrasound data acquired using a medical device inserted into the patient; (3) dynamically integrates the current anatomical data with the multi-dimensional ultrasound data to facilitate localization of a position of the medical device internal to the patient; and (4) visually depicts the localized position of the medical device internal to the patient on a display.

In yet another embodiment, a computer-readable medium provides instructions executable on a computer. The instructions direct receiving current three-dimensional computed tomography data; receiving real-time three-dimensional ultrasound data acquired via a medical device during an intervention; dynamically fusing the real-time three-dimensional ultrasound data with the current three-dimensional computed tomography data during the intervention to localize a current position of the medical device within a patient; and displaying the current position of the medical device in relation to an anatomical structure of the patient.

Advantages will become more apparent to those skilled in the art from the following description of the preferred embodiments which have been shown and described by way of illustration. As will be realized, the system and method are capable of other and different embodiments, and their details are capable of modification in various respects. Accordingly, the drawings and description are to be regarded as illustrative in nature and not as restrictive.

### BRIEF DESCRIPTION OF THE DRAWINGS

FIG. 1 illustrates an exemplary medical workflow;

FIG. 2 illustrates another exemplary medical workflow;

FIG. 3 illustrates an exemplary catheter with a multi-dimensional array of sensors; and

FIG. 4 illustrates an exemplary data processor configured or adapted to provide the functionality associated with the medical workflows discussed herein.

### DETAILED DESCRIPTION

The embodiments described herein include methods, processes, apparatuses, instructions, or systems related to enhanced medical workflows. The workflows may (1) acquire current anatomical data as a patient is on an operating table for an interventional medical procedure, and (2) generate an electroanatomical (EA) map of a portion of the patient via during the intervention. The EA map may be generated from data acquired using a medical device inserted into the patient during the intervention. Subsequently, (3) the anatomical data may be dynamically fused/integrated with the EA map in real-time or with minimum time delay. The fused data may be displayed and/or used to automatically or manually accurately localize a position of the medical device or a portion thereof within the patient during the intervention.

In one aspect, the workflow may be associated with an a-fib or other ablation procedure performed in an electrophysiology (EP) lab. Anatomical data may be acquired in the EP lab via computed tomography, magnetic resonance, or other medical imaging procedures. The EA map may be generated from ultrasound or other data. In one embodiment, the anatomical data may be cardiac computed tomography data acquired using a C-arm based angiography system and the ultrasound data may be acquired using an enhanced catheter employing a multi-dimensional and/or forward looking array of ultrasound sensors or emitters.

In sum, the present embodiments may use current anatomical data in conjunction with a more accurate EA map (based upon multi-dimensional ultrasound data) to alleviate inefficiencies and inaccuracies associated with conventional medical workflows. As a result, the present embodiments may shorten conventional interventional workflows, eliminate unnecessary procedures, alleviate inconveniences to the patient, enhance safety, improve operational success rates, and/or reduce costs.

### I. Ablation Procedures

In general, electricity normally flows throughout the heart in a regular pattern to facilitate heart muscle contractions. However, sometimes the electrical flow gets interrupted, disturbing normal heart rhythms. One type of treatment for heart rhythm disorders is called ablation.

Cardiac ablation may be used to treat rapid heartbeats that begin in the upper chambers, or atria, of the heart. Specific types of heart rhythm disorders include supraventricular tachycardias, atrial fibrillation, atrial flutter, AV nodal reentrant tachycardia, AV reentrant tachycardia, or atrial tachycardia.

With atrial fibrillation (a-fib), the upper part of the heart beats more rapidly than the rest of the heart. The top part of the heart may quiver rapidly and irregularly (fibrillate) hundreds of times a minute.

Less frequently, ablation may be used to treat heart rhythm disorders associated with the heart's lower chambers, known as the ventricles. For instance, ventricular tachycardia may cause sudden cardiac death.

Nowadays, an ablation procedure may be relatively non-invasive and performed in an electrophysiology (EP) lab. The procedure may involve inserting a catheter into a blood vessel of the patient and guiding the catheter via a narrow, flexible guide wire into the heart. The catheter may be guided from the entry point into the patient to the heart using images created by a fluoroscope (x-ray related device) that provides continuous images of the catheter.

Once the catheter reaches the heart, electrodes near the tip of the catheter may gather data and a variety of electrical measurements may be calculated. The measurements may be used to identify where the faulty electrical site is located within the heart. This process is referred to as “electrical mapping.” Once the problematic area is located, damaged tissue may be destroyed to eliminate the electrical disturbance.

### II. Exemplary EP Lab

The present embodiments may involve acquiring current anatomical data for real-time integration with ultrasound data or other electroanatomical mapping data. In one embodiment, a modified EP lab may be operable to obtain CT data via a C-arm system, perform 3D echocardiography, dynamically fuse the 3D anatomical data with the 3D echo data, and provide for the real-time detection and/or calculation of the location of a medical device, such as catheter, within the patient using the 3D echo data.

Conventionally, the planning of procedures such as atrial fibrillation ablations may have been based upon CT or MR images acquired before the examination or intervention. However, morphologic information of these pre-procedural images may be limited by the time lag to the actual intervention. The pre-procedural approach may not account for dynamic changes that develop in the anatomical structures with time. The present embodiments may permit cross-sectional images to be acquired in the EP lab during real-time or almost real-time to guide the intervention.

For example, cross-sectional CT images may be acquired in the EP lab as an initial step of an ablation or other intervention. Compared to pre-procedural CT or MR images, the CT images may be used to visualize the anatomy of the patient's left atrium in its actual state during the intervention. As a result, reliable orientation of images of the patient's anatomy during the intervention may be facilitated. Additionally, the patient may be spared the inconvenience of having to undergo a separate imaging procedure.

In one aspect, the workflow may involve electrocardiogram (ECG) triggered rotational angiography and retrospective gated three-dimensional reconstruction. The ECG triggering of the temporal image resolution may permit enhanced visualization of the moving structures in multiple dimensions. The EP lab also may be associated with one or more user interfaces that provide access to and display DICOM (Digital Imaging and Communications in Medicine) information.

An efficient and complete workflow may be performed within a modified EP lab. In one embodiment, the EP lab may facilitate a workflow that includes (1) the use of Cardiac Dyna CT™ (currently undergoing clinical evaluations) or other medical equipment to obtain CT or other three-dimensional (3D) anatomical data sets of the patient's anatomy when the patient is on a medical table within the EP lab; (2) the use of 3D ultrasound data (such as echocardiography data), which may be intra-cardiac echocardiography (ICE) or extra-corporal data (such as transthoracic echocardiogram (TTE) or transesophageal echocardiogram (TEE) data); and (3) registration/fusion of the 3D anatomical data and the 3D echocardiography data. Algorithms for this type of 3D data/3D data fusion are known to software and programming experts within the field of image processing.

The EP lab may facilitate a workflow that also includes (4) the use of real-time 3D echocardiography to localize a catheter, particularly the tip of the catheter for EA mapping; (5) miniaturized ultrasound microprocessor based probes or sensors (preferably, silicon based sensors) mounted on the catheter; and (6) through the registration of the EA map with the anatomical data, determining and displaying the localized position of the catheter (tip) relative to the CT or other anatomical data. The use of current anatomical data and/or more accurate ultrasound data may enhance the accuracy of the localized position of the catheter determined and displayed as compared to conventional workflows. The workflow may further include (7) measuring an EA map of the atrium that may be related to the CT or other anatomical data; (8) during an ablation procedure, determining the location of the catheter again from real-time 3D echocardiography data and relating the current location of the catheter to the patient's anatomy and the EA map; and (9) performing ablations in the EP lab based on only anatomical or electroanatomical data, or a combination of the two.

The workflow steps identified above are representative, but not exclusive. Other variants and combinations may be used. As a result, the workflow may include additional, fewer, or alternate steps. Additionally, the workflow may not be limited to ablation procedures. The workflow may be a cardiac related workflow, such as a valve replacement, or an abdomen, liver, cancer, or tumor related procedure, and/or another medical workflow.

Therefore, the present workflows may combine real-time anatomical data, with three-dimensional ultrasound data and catheter location data, and measure electrical signals for the heart. The anatomical data may be fused with the ultrasound data in real-time during the intervention, such as to accurately represent the current position of the catheter tip within the patient. The workflow may involve combining x-ray anatomical data, which provides a global view, with ultrasound data, which provides a more limited field of view, such as a real-time localized view.

In a preferred embodiment, a method and system may acquire three-dimensional images of a patient's anatomy, such as via DynaCT™ or other imaging systems. The three-dimensional anatomical images may be dynamically fused with real-time three-dimensional ultrasound images. The real-time three-dimensional ultrasound may be used to detect and localize a medical device within a patient during an intervention. The device detected using an ultrasound technique may be depicted relative to the three-dimensional anatomical images. In one aspect, the medical device is a mapping catheter (“EP”). The mapping catheter may facilitate the generation of an EA map solely from the measured electrical potential and the known location, without the aid of any other localization sensors.

### III. Exemplary Workflow

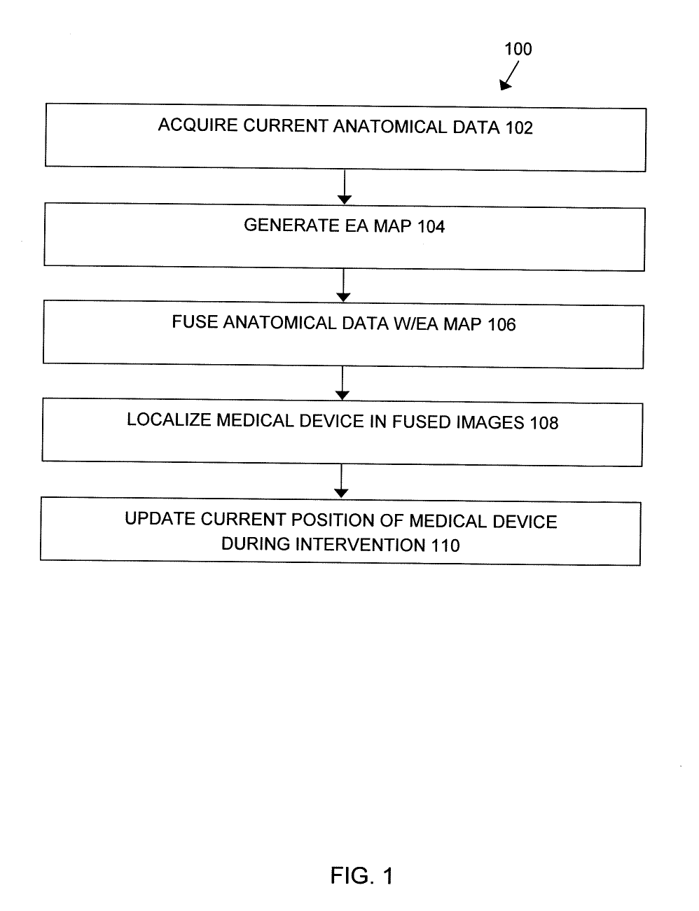

FIG. 1 illustrates an exemplary workflow. The workflow 100 may include, during or as a part of an intervention, acquiring current anatomical data of a patient 102, generating an electroanatomical map of the patient 104, dynamically fusing the current anatomical data and the electroanatomical map to create fused images 106, localizing a medical device in relation to the fused images 108, and updating the current position of the medical device during the intervention 110. The workflow may include additional, fewer, or alternate steps.

The workflow 100 may include acquiring current anatomical data of a patient 102. The anatomical data may be acquired as part of an interventional procedure, such as when the patient is on a medical table at the initial stages of the procedure or during the procedure. For example, the anatomical data may be acquired either just before or during the intervention. The anatomical data also may be updated during the intervention.

A number of imaging procedures may be used to acquire the anatomical data. The medical imaging equipment, preferably located within or near the modified EP lab, may relate to processing images illustrating an enhanced region of interest within a patient. For example, various types of contrast medium may be administered to a medical patient. The contrast mediums enhance the scans acquired by scanning a patient or images of the patient, the scans and images may be recorded by an external recording device as enhancement data. The contrast medium typically travels through a portion of the body, such as in the blood stream, and reaches an area that medical personnel are interested in analyzing. While the contrast medium is traveling through or collected within a region of interest, a series of scans or images of the region of interest of the patient may be recorded for processing and display by software applications. The enhanced region of interest may show the brain, the abdomen, the heart, the liver, a lung, a breast, the head, a limb or any other body area.

The image data may be generated by one or more specific type of image processes that are used to produce the images or scans of the patient. In general, the types of imaging processes performed by the medical equipment being used to produce patient images or scans of internal regions of interest include radiography, angioplasty, computerized tomography, ultrasound and magnetic resonance imaging (MRI). Additional types of imaging processes may performed by the medical equipment, such as perfusion and diffusion weighted MRI, cardiac computed tomography, computerized axial tomographic scan, electron-beam computed tomography, radionuclide imaging, radionuclide angiography, single photon emission computed tomography (SPECT), cardiac positron emission tomography (PET), digital cardiac angiography (DSA), and digital subtraction angiography (DSA). Alternate imaging processes may be used.

The workflow 100 may include generating an EA map of the patient 104. The EA map may be generated using data acquired from a medical device inserted into the patient during the intervention. The medical device may be a catheter, such as a catheter having a multi-dimensional array of ultrasound sensors as disclosed herein. Other exemplary catheters that may be used are disclosed by U.S. Pat. Nos. 5,947,905 and 5,771,895, which are both incorporated herein by reference in their entireties. Other medical devices may be used, such as modified wires and needles.

The electrical mapping may be performed using a procedure referred to as echocardiography, such as 3D echocardiography. 3D echocardiography may be based upon 2D techniques and use the same types of tools. By tracking the size, shape, and position of heart structures in dozens of imaging planes aligned in three dimensions via common reference points, the entire heart may be reconstructed as a solid object with accurate representation of its shape. Known techniques for 3D echocardiography may model the left and right ventricles, the left and right atria, and other portions of the heart. Other organs, such as the liver, as well as muscles and vessels may be modeled. From the reconstruction of anatomical structures, 3D echocardiography may allow medical personnel to ascertain the size and shape of the structures, and the spatial relationships between them. Other electrical mapping procedures may be used.

In one aspect, generating the EA map may be accomplished as part of ultrasound imaging technique. Exemplary methods of ultrasound imaging and image reconstruction are disclosed by U.S. Pat. Nos. 5,787,889; 5,934,288; and 6,139,500, which are all incorporated herein by reference in their entireties. Other imaging techniques may be used.

The workflow 100 may include dynamically fusing the current anatomical data and the EA map 106. The fusion of the current anatomical data and the EA map may create composite images. The fusion may be accomplished by techniques known to programming experts in the field. The composite images created may be used to localize the position of the medical device within the patient. In one aspect, the fusion of the current anatomical data and the EA mapping data may be accomplished using a common coordinate system. The position of the medical device may then be localized with respect to the common coordinate system. An exemplary fusion technique is disclosed by U.S. Pat. No. 6,019,724, which is incorporated herein by reference in its entirety. Other fusion techniques may be used.

The workflow 100 may include localizing a medical device in the fused images 108. The localization of the medical device may be performed automatically by a processor. For instance, a processor may be able to calculate a localized position of the medical device in relation to either the anatomical date or EA map, or a combination of the two. The calculation may be done with more precision than conventional techniques as the anatomical data of the present embodiments is more current and/or accurate. Moreover, the electroanatomical data may be more accurate if acquired with the enhanced catheters or other medical devices as described herein. As noted above, the position of the medical device may be localized with respect to a common coordinate system of the fused images. Other automatic localization techniques may be used.

By displaying the composite images of the fused current anatomical data and the electroanatomical map with a visual depiction, outline, or an actual or virtual image of the medical device, medical personnel may then be able to visually ascertain or localize the position of the medical device in relation to the patient's anatomy, with images of the patient's anatomy being reproduced on the display from the anatomical data.

In one aspect, the composite images may be a fusion of solely the anatomical data and the EA map. A visual depiction of the medical device may then be superimposed upon the registration of the EA map with anatomical image data by a processor. Alternatively, the composite images themselves may include a visual depiction, either actual or virtual, of the medical device. Either manner permits localization of the medical device and/or display of a localized position of the medical device in relation to one or more anatomical structures of the patient.

The workflow 100 may include updating the current position of the medical device displayed during the intervention 110. As noted above, the composite images displayed may show or be altered to show a localized position of the medical device within the patient during the interventional procedure. As the intervention progresses, the localized position of the medical device may be recalculated, such as with respect to the common coordinate system. As a result, medical personnel may be presented with a more accurate localization of the current position of the medical device internal to the patient in real-time during the intervention.

For instance, a visual depiction of the medical device may be moved on the display to new and updated coordinates to display the medical device in relation to the fused or composite images depicting the registration of the EA map with the anatomical data. Alternatively, the fused or composite images may be altered. The current position of the medical device being displayed may be updated in other manners.

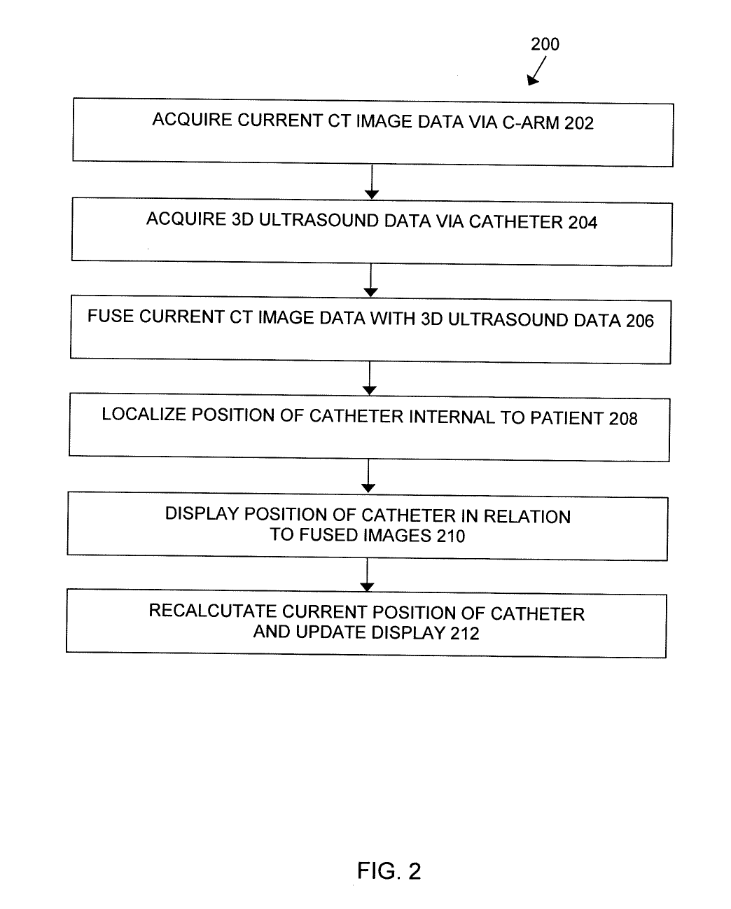

FIG. 2 illustrates another exemplary workflow 200. The workflow 200 may include acquiring current 3D CT image data via a C-arm based system 202, acquiring 3D ultrasound data via a catheter having a multi-dimensional array of sensors 204, fusing the current 3D CT image data with the 3D ultrasound data 206, localizing a position of the catheter internal to the patient 208, displaying the localized position of the catheter in relation to the fused images 210, and recalculating the current position of the catheter during the intervention and updating the display to reflect the recalculated current position of the catheter 212. The workflow may include additional, fewer, or alternate actions.

### IV. Exemplary Catheter

The present embodiments also relate to an enhanced ultrasound catheter with a forward-orientated field of view. Typical endovascular interventions may be performed using radiation. Contrast agent may be injected selectively to make the vessels of interest visible. However, a disadvantage may be that only the open lumen of the vessel is shown. As a result, the nature of the vessel wall may only be assessed indirectly.

To overcome this disadvantage, intravascular ultrasound (IVUS) may be employed. Optical methods, such as OCT (optical coherence tomography) have also been tried in the clinical research field. A disadvantage of these methods may be that the radiation is emitted perpendicular to the tip of the catheter. This may result in acquiring data associated with only a “slice” of the vessel wall at the point of the catheter tip.

The present embodiments provide an enhanced medical device operable to acquire a more complete and forward-looking image. For instance, the medical device, if used during an endovascular or other intervention, may provide the physician with a view forward of the endoscope. The nature of the vessel wall (calcification, fibrous plaques, lipid plaque, etc.) in front of the medical device, such as a catheter or guide wire, may be shown on a display.

Although conventional optical angioscopy was an attempt at improving images displayed, it did not prove overly useful for the current applications as light (visible or infrared) cannot penetrate through blood. Therefore, optical angioscopy may require rinsing the vessel to be free of blood, for instance with physiological saline solution. Accordingly, the practical application of optical angioscopy may be overly inconvenient and involve too many complications.

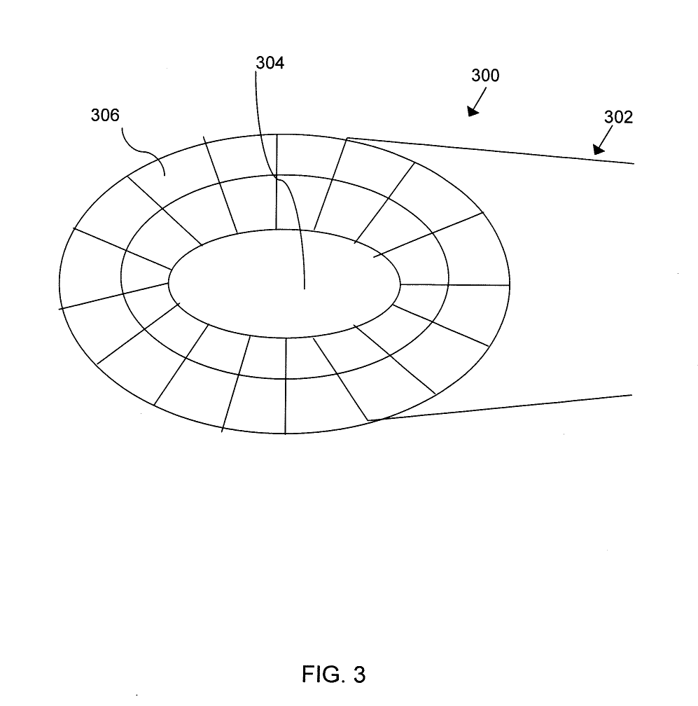

On the other hand, ultrasound does not have the limitations associated with optical angioscopy as ultrasound may easily “look” through blood. The most recent developments based on semiconductors (sometimes referred to as ultrasound on silicon) may produce miniature ultrasound emitters and receivers with highly variable geometries. The enhanced medical devices discussed herein may attach the tiniest ultrasound probes at the tip of the catheter along its circumference (such as shown in FIG. 3) or on its nose. In one embodiment, the individual ultrasound emitters function as forward-pointing sound wedges. By targeted electronic triggering of the individual silicon elements, the shape and range of the ultrasound field may be varied and adjusted. Additionally, using virtual histology, such as that produced by equipment available from Volcano Corporation™, it is now known that the backscattered ultrasound signals may be processed in such a way to differentiate among the various plaques on the vessel walls.

FIG. 3 shows an exemplary enhanced catheter 300. The catheter 300 may include a long, slender body 302, an internal lumen 304, and an array of ultrasound emitters 306. The catheter may include additional, fewer, or alternate components.

The catheter 300 may include a long and narrow body 302, such as those known in the art. The internal lumen 304 may be used to house a guide wire. The guide wire may be used to navigate the catheter 300 internal to the patient during the intervention.

The array 306 may include a number of ultrasound emitters and/or sensors. The array 306 may be a multi-dimensional array, “looking” in a number of directions. The array 306 may be positioned on the circumference of the catheter 300, such as near the distal end. Alternatively, the array 306 may be positioned on the tip or forward most point of the catheter 300 itself. The array 306 may include ultrasound sensors encompassing a rounded forward tip of the catheter 300 and further extending to encompass a portion of the rounded circumference of the longitudinal body of the catheter 300. Other arrangements may be used.

In one aspect, the array 306 may be a multi-dimensional forward looking array mounted on the tip or the body of the catheter. The array 306 may gather data as the catheter is advanced into the patient. In one embodiment, the array 306 may comprise a plurality of sensors arranged similar to a sonar dome on the bow of a ship or submarine. Of course, the sensors themselves would be much smaller in size to accommodate medical applications, instead of nautical uses. Other geometries and arrangements of ultrasound sensors may be used.

The ultrasound catheter 300 shown in FIG. 3 may have a hollow lumen through which a thin guide wire can be advanced. In practical use, the forward-oriented ultrasound imaging may be used to control the advancement of the guide wire relative to the vessel wall and to possible lesions.

In one aspect, the ultrasound catheter may be used during a medical application directed toward the reopening of chronic total occlusions (CTOs). The ultrasound catheter may be advanced as far as the proximal end of the occlusion. The ultrasound image may show the user the composition of the thrombus (soft/hard or calcified) and any open micro-channels that may be present. The guide wire may then be advanced in a targeted way through the soft plaque components or through the microscopic channels.

In another aspect, the guide wire may be navigated magnetically. By magnetically navigating the guide wire, the method may be further automated. From the ultrasound image, the point where the advancement of the wire is easiest and involves the least risk may be identified. The extreme magnetic field may be adjusted such that the tip of the guide wire is steered into that point. An incremental advancement may then be performed manually or automatically. The process may be repeated until the occlusion has been pierced completely.

The catheter tip may have a known and characteristic form. As a result, the catheter may be visually or graphically modeled to enhance the images displayed. The characteristic form of the catheter tip may provide for a definite allocation of the catheter within the ultrasound image.

The catheter of FIG. 3 also may include a sensor system, such as a sensor system for measuring electrical signals from the heart. A carto sensor on the tip of the catheter device may record signals for mapping the medical device onto the anatomy of the patient. With the ultrasound catheter, monitoring may be performed in real-time without exposing the patient to ionizing radiation.

### V. Exemplary Data Processing System

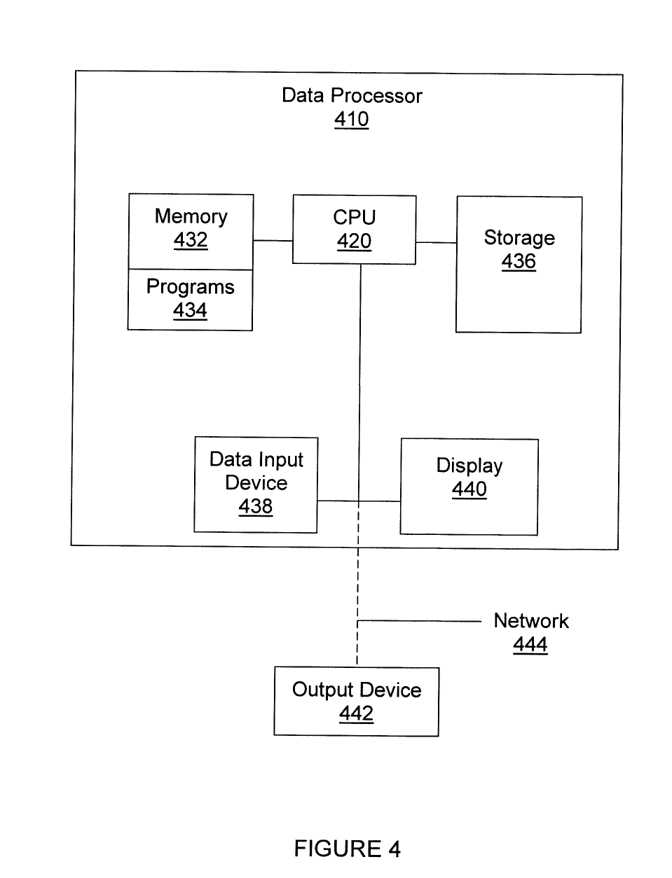

FIG. 4 illustrates an exemplary data processor 410 configured or adapted to provide the functionality for the workflows as discussed herein. The data processor 410 may be located at a central location, such as within or near an EP lab. The data processor may include a central processing unit (CPU) 420, a memory 432, a storage device 436, a data input device 438, and a display 440. The processor 410 also may have an external output device 442, which may be a display, a monitor, a printer or a communications port. The processor 410 may be a personal computer, work station, PACS station, or other medical imaging system. The processor 410 may be interconnected to a network 444, such as an intranet, the Internet, or an intranet connected to the Internet. The processor 410 may be interconnected to a customer system or a remote location via the network 444. The data processor 410 is provided for descriptive purposes and is not intended to limit the scope of the present system. The processor may have additional, fewer, or alternate components.

A program 434 may reside on the memory 432 and include one or more sequences of executable code or coded instructions that are executed by the CPU 420. The program 434 may be loaded into the memory 432 from the storage device 436. The CPU 420 may execute one or more sequences of instructions of the program 434 to process data. Data may be input to the data processor 410 with the data input device 438 and/or received from the network 444 or customer system. The program 434 may interface the data input device 438 and/or the network 444 or customer system for the input of data. Data processed by the data processor 410 may be provided as an output to the display 440, the external output device 442, the network 444, the customer system, and/or stored in a database.

The program 434 and other data may be stored on or read from machine-readable medium, including secondary storage devices such as hard disks, floppy disks, CD-ROMS, and DVDs; electromagnetic signals; or other forms of machine readable medium, either currently known or later developed. The program 434, memory 432, and other data may comprise and store a database related to medical images of the patient.

The data processor 410 may be operable to acquire current anatomical data, such as data acquired via a C-arm imaging system, generate an EA map, and fuse the current anatomical data with the EA map. For instance, the data processor 410 may (1) receive current anatomical data of a patient; (2) receive multi-dimensional ultrasound data acquired using a medical device inserted into the patient; (3) dynamically integrate the current anatomical data with the multi-dimensional ultrasound data to facilitate localization of a position of the medical device internal to the patient; and (4) visually depict the localized position of the medical device internal to the patient on a display.

The program or other software associated with the data processor system may include instructions that direct the fusion of anatomical data with an EA map to create composite images and localizing a medical device being used during an intervention within the composite images. In one aspect, the instructions may direct receiving current three-dimensional computed tomography data; receiving real-time three-dimensional ultrasound data acquired via a medical device during an intervention; dynamically fusing the real-time three-dimensional ultrasound data with the current three-dimensional computed tomography data during the intervention to localize a current position of the medical device within a patient; and displaying the current position of the medical device in relation to an anatomical structure of the patient.

The data processor 410 may facilitate an interventional workflow as discussed herein. The data processor 410 may provide functionality related to standard x-ray fluoroscopy and carto systems, as well as accept data from sensors integrated into a tip of a catheter or other medical device. The data processor 410 may generate an EA map as the catheter is advanced within a patient and measure the local potential.

The data processor 410 also may perform three-dimensional reconstruction of anatomical structures and provide rotational imaging for navigating the catheter within a patient. The navigation may be facilitated by true and accurate representation of the anatomical structure(s). Additionally, the data processor 410 may display the representation of the anatomical structure in real-time or dynamically, such as when the catheter is inside of the left atrium of the patient. In other words, the data processor 410 may combine real-time ultrasound image data with updated anatomical data to create an up-to-date integrated display. The up-to-date integrated display may facilitate the navigation of medical devices within the patient.

The data processor 410 may combine 2D or 3D ultrasound data with volume and image anatomical data dynamically acquired or stored in a memory. For instance, the ultrasound data may be provided with a “slice” or specific view of the patient. If the ultrasound data is combined with volume information, accurate localization of the catheter or other medical instrument may be facilitated.

In sum, as discussed elsewhere herein, in a preferred embodiment, a method and system may acquire three-dimensional images of a patient's anatomy, such as via DynaCT™ or other imaging systems. The three-dimensional anatomical images may be dynamically fused with real-time three-dimensional ultrasound images. The real-time three-dimensional ultrasound may be used to detect and localize a medical device within a patient during an intervention. The device detected using an ultrasound technique may be depicted relative to the three-dimensional anatomical images. In one aspect, the medical device is a mapping catheter (“EP”) such that an EA map may be generated from the measured electrical potential and the known location, without any other localization sensors (e.g., Carto™ system of Biosense Webster™).

While the preferred embodiments of the invention have been described, it should be understood that the invention is not so limited and modifications may be made without departing from the invention. The scope of the invention is defined by the appended claims, and all devices that come within the meaning of the claims, either literally or by equivalence, are intended to be embraced therein.

It is therefore intended that the foregoing detailed description be regarded as illustrative rather than limiting, and that it be understood that it is the following claims, including all equivalents, that are intended to define the spirit and scope of this invention.

What is claimed is:

1. A medical method for assisting, with a system, an interventional procedure, the method comprising:
as part of the interventional medical procedure:
(1) acquiring current anatomical data of a patient via a medical imaging device;
(2) generating a current electroanatomical map of the patient;
(3) dynamically fusing the current anatomical data with the current electroanatomical map; and
(4) displaying fused images associated with the fusion of the current anatomical data with the current electroanatomical map in real-time such that the fused images displayed facilitate completion of the interventional medical procedure.

2. The method of claim 1, the medical imaging device being a C-arm computed tomography imaging device.

3. The method of claim 1, the electroanatomical map being generated from three-dimensional ultrasound data.

4. The method of claim 1, the electroanatomical map being generated from an intra-cardiac or extra-corporal data set.

5. The method of claim 1, the workflow further comprising using the fused images to localize a tip of a catheter internal to the patient during the interventional medical procedure.

6. The method of claim 1, the current electroanatomical map being generated from multi-dimensional ultrasound data acquired by a catheter inserted into the patient during the interventional medical procedure, the catheter acquires the multi-dimensional ultrasound data using a multi-dimensional looking array of ultrasound sensors.

7. The method of claim 1, the workflow comprising:
displaying a depiction of a medical device used to generate the electroanatomical map in relation to the fused images; and
during the interventional medical procedure, recalculating a current position of the medical device and updating the display of the fused images to illustrate the current position of the medical device internal to the patient.

8. The method of claim 1, the interventional medical procedure being an ablation procedure.

9. A medical method for assisting, with a system, an interventional procedure, the method comprising:
acquiring current anatomical data of a patient, as part of an interventional procedure, when the patient is on a medical table;
generating an electroanatomical map of the patient using data acquired from a medical device inserted into the patient during the interventional procedure;
dynamically fusing the current anatomical data and the electroanatomical map to create composite images;
localizing a position of the medical device within the patient in relation to the composite images; and
displaying the composite images associated with the fused current anatomical data and the electroanatomical map such that the localized position of the medical device within the patient during the interventional procedure may be ascertained.

10. The method of claim 9, the interventional procedure being an ablation procedure and the medical device being a mapping catheter that facilitates the generation of the electroanatomical map from the measured electrical potential and the known location of the medical device without the aid of additional localization sensors.

11. The method of claim 9, the current anatomical data comprising cardiac computed tomography data acquired using a C-arm based system.

12. The method of claim 9, the electroanatomical map being generated from multi-dimensional ultrasound data acquired via the medical device, the medical device being a catheter.

13. The method of claim 12, the catheter having a multi-dimensional looking array of ultrasound sensors.

14. The method of claim 13, the multi-dimensional ultrasound data is intra-cardiac or extra-corporal data.

15. The method of claim 9, the workflow comprising:
recalculating a current position of the medical device during the interventional procedure; and
dynamically altering the composite images to display the current position of the medical device.

16. A data processing system for facilitating an interventional procedure, the system comprising:
a processing unit that (1) receives current anatomical data of a patient; (2) receives multi-dimensional ultrasound data acquired using a medical device inserted into the patient; (3) dynamically integrates the current anatomical data with the multi-dimensional ultrasound data to facilitate localization of a position of the medical device internal to the patient; and (4) visually depicts the localized position of the medical device internal to the patient on a display.

17. The system of claim 16, the anatomical data being computed tomography data.

18. The system of claim 16, the medical device being a catheter having a multi-dimensional forward-looking array of sensors.

19. The system of claim 16, the processor updating the localized position of the medical device being displayed in real-time as the medical device is moved within the patient.

20. A computer-readable medium having instructions executable on a computer stored thereon, the instructions comprising:
receiving current three-dimensional computed tomography data;
receiving real-time three-dimensional ultrasound data acquired via a medical device during an intervention;
dynamically fusing the real-time three-dimensional ultrasound data with the current three-dimensional computed tomography data during the intervention to localize a current position of the medical device within a patient; and
displaying the current position of the medical device in relation to an anatomical structure of the patient.

21. The computer-readable medium of claim 20, the medical device being a catheter having a multi-dimensional array of ultrasound sensors.
